(19) United States
(12) Patent Application Publication
(10) Pub. No.: US 2005/0026196 A1
(43) Pub. Date: Feb. 3, 2005

(54) Prognostic marker for prostate cancer and methods and kits for using same

(75) Inventors: Fred Saad, (US); Anne-Marie Mes-Masson, (US); Laurent Lessard, (US)

Correspondence Address: GOUDREAU GAGE DUBUC, 800 PLACE VICTORIA, SUITE 3400, MONTREAL, QUEBEC, H4Z 1E9, CA

(73) Assignees: Centre Hospitalier Universitaire de Montreal; Universite de Montreal

(21) Appl. No.: 10/874,413

(22) Filed: Jun. 23, 2004

Related U.S. Application Data
(60) Provisional application No. 60/480,216, filed on Jun. 23, 2003.

(51) Int. Cl. C12Q 1/68; G01N 33/574; G06F 19/00; G01N 33/48; G01N 33/50

(52) U.S. Cl. 435/6; 435/7.23; 702/19

(57) ABSTRACT

A method for predicting whether a patient suffering from a prostate cancer is at risk of experiencing a cancer progression or recurrence which comprises the steps of : obtaining a Gleason score of said prostate tumor sample; assessing the proportion of NF-kB localized in the nuclei of said tumor sample with regard to all NF-kB present in said tumor sample.

### FIELD OF THE INVENTION

The present invention relates to a prognostic marker for prostate cancer and methods and kits for using same.

### BACKGROUND OF THE INVENTION

Prostate cancer is the most frequently diagnosed cancer and the second leading cause of cancer death in North American men. It is difficult to predict whether a clinically localized prostate cancer will remain latent or progress to an aggressive, highly metastatic disease. In fact, molecular events associated with carcinogenesis and tumor progression in prostate cancer are still unclear. There is a need for molecular markers that could help predict the aggressiveness of prostate cancers. The degree of histological differentiation (Gleason grade) is the best marker known to date, but the prognosis is difficult to establish in the case of moderately differentiated tissues (Gleason scores 5 to 7) which represent more than two thirds of diagnosed cancers.

The NF-κB transcription factor family is composed of 5 structurally related members that possess a N-terminal Rel-Homology (RH) domain involved in protein-protein interactions and DNA-binding. NF-κB proteins can be divided into 2 classes based on sequences C-terminal to their RH domain. The first class includes Rel (c-Rel), RelA (p65), and RelB proteins that are characterized by a C-terminal transactivation domain. NF-κB1 (p50 and its precursor p105) and NF-κB2 (p52 and its precursor p100) form the second class; the precursors contain inhibitory C-terminal ankyrin repeats that are cleaved to create transcriptionally active p50 and p52 proteins. Rel/NF-κB proteins exist as homo- and heterodimers. The canonical (RelA/p50) and non-canonical (RelB/p52) NF-κB complexes are expressed in many cells but kept inactive and maintained in the cytoplasm by IκB inhibitors or the p100 precursor respectively. Normally, the activation of NF-κB requires signals that converge to IκB kinases (IKKs). In the canonical pathway the IKK complex (IKKα, β, γ) phosphorylates IκBs (IκBα or IκBβ) which are then ubiquitinated and targeted for proteasome-dependent degradation. In the non-canonical pathway, IKKα dimers regulate the processing of the p100 precursor. Subsequently, NF-κB dimers translocate to the nucleus and activate the expression of various genes involved in cell growth, differentiation, inflammatory responses and the regulation of apoptosis.

NF-κB pathways and genes are frequently altered in lymphoid and non-lymphoid cancers. For instance, chromosomal alterations involving the c-rel and NF-κB2 genes are have been detected in several B- and T-cell lymphomas. Constitutively nuclear and active RelA/p50 dimers can prevent cell death by apoptosis in many cancer cell types after chemotherapy, radiotherapy or TNF-α treatment. More recently, constitutive activation of NF-κB (RelA/p50) has been detected in androgen-independent prostate cancer cell lines as well as in prostate cancer tissues and appears to promote cell growth, survival, and metastasis.

While a role for NF-κB in prostate cancer is being considered, the prognostic significance of NF-κB in human prostate cancer tissues has not been investigated.

The present invention seeks to meet these needs and other needs.

The present description refers to a number of documents, the content of which is herein incorporated by reference in their entirety.

### SUMMARY OF THE INVENTION

There are no known molecular markers that can predict prostate cancer progression. The degree of histological differentiation, the Gleason grade, is the most reliable prognostic marker. However, clinically-confined tumors of intermediate Gleason grade, which represent two thirds of diagnosed cancers, can either remain latent or progress to an aggressive malignancy. The present invention relates to the use of NF-κB as a molecular marker alone and in combination with other prognostic markers such as the Gleason grade. Indeed, the expression and sub-cellular localization of NF-κB, detected by means such as in situ revealing methods such as immunohistochemistry on prostate biopsy and radical prostatectomy specimens, can be used as a molecular marker to predict prostate cancer progression. NF-κB can indicate the presence or predict the development of metastases to tissues like lymph nodes and bones, and/or biochemical relapse, and/or development of hormone-refractory tumors.

In accordance with the present invention, there is provided a method for predicting the risk incurred by a patient suffering of a prostate cancer of experiencing a cancer progression or recurrence, which comprises the steps of: staining the cells and the nuclei of a prostate tumor sample obtained from said patient; staining the cells and the nuclei for the presence of NF-kB; calculating a proportion of staining of NF-kB localized in the nuclei with regard to the total staining of NF-kB in a given surface area of tumor sample, wherein a risk of cancer progression or recurrence is recurrence when 5% or more (more specifically 10% or more, even more specifically 20% or more) of the NF-kB staining in said surface is localized in the cell nuclei. Any result equal to or higher than this cut-off value is considered a positive and significant nuclear staining of NF-kB. This cut-off value may be used alone or in combination with the currently used Gleason score. Particularly, a Gleason score of at least 5, namely 5-7 and 8-10, along with a positive nuclear staining of NF-KB provides for an accurate prognosis.

In accordance with the present invention, there is provided a method for predicting whether a patient suffering from a prostate cancer is at risk of experiencing a cancer progression or recurrence which comprises the steps of: obtaining a Gleason score of said prostate tumor sample; assessing the proportion of NF-kB localized in the nuclei of said tumor sample with regard to all NF-kB present in said tumor sample.

According to an aspect of the invention, there is provided a method for preparing a prostate cancer sample for predicting whether a patient suffering from a prostate cancer is at risk of experiencing a cancer progression or recurrence which comprises the steps of: obtaining a Gleason score of said prostate tumor sample; staining the cells and the nuclei of a prostate tumor sample obtained from said patient; staining the cells and the nuclei for the presence of NF-kB. In a specific embodiment, the method further comprises calculating a proportion of staining of NF-kB localized in the nuclei with regard to the total cell staining of NF-kB in a given surface area of tumor sample.

According to an other aspect of the invention, there is provided a method for predicting whether a patient suffering from a prostate cancer is at risk of experiencing a cancer progression or recurrence which comprises the steps of: staining the cells and the nuclei of a prostate tumor sample obtained from said patient; obtaining a Gleason score of said prostate tumor sample; staining the cells and the nuclei for the presence of NF-kB; calculating a proportion of staining of NF-kB localized in the nuclei with regard to the total cell staining of NF-kB in a given surface area of tumor sample, wherein about 5% or more of the NF-kB staining in said surface being localized in the cell nuclei and a Gleason score higher than 5 are indicative that the patient is at risk of experiencing cancer progression or recurrence. In a specific embodiment, about 10% or more of the NF-kB staining in said surface localized in the cell nuclei is indicative that the patient is at risk of experiencing cancer progression or recurrence. In an other specific embodiment, about 20% or more of the NF-kB staining in said surface localized in the cell nuclei is indicative that the patient is at risk of experiencing cancer progression or recurrence. In other specific embodiments, the cancer progression or recurrence is identified through biochemical recurrence, lymph node metastases, bone metastases, and/or hormone refractoriness. In an other specific embodiment, the presence of NF-kB is revealed with a ligand to NF-kB p65 epitope. In an other specific embodiment, the ligand is a monoclonal antibody specific to the C-terminal region of p65.

According to an other. aspect of the invention, there is provided a diagnostic kit for predicting a risk incurred by a patient suffering from a prostate cancer of experiencing a cancer progression or recurrence, which comprises: reactants for staining the cells and nuclei of a prostate tumor sample, and reactants for staining the cells and nuclei for the presence of NF-kB. In a specific embodiment of the kit, the reactants for staining the cells and nuclei for the presence of NF-kB comprise a ligand to NF-KB p65 epitope. In a more specific embodiment, the ligand is a monoclonal antibody to the C-terminal region of p65.

As used herein, the terminology “cancer progression or recurrence” refers to the development or spread (in size or malignancy) of an existing tumor or appearance of new symptoms and tumors. Without being so limited, this terminology refers to lymph node metastases, bone metastases, biochemical recurrence and/or hormone refractoriness for instance.

As used herein, the terminology “significant risk of cancer progression or recurrence” cannot be defined quantitatively. It refers to a probability of experiencing cancer progression or recurrence that is higher than the probability of not experiencing same.

This invention will be described herein below, by reference to specific examples, embodiments and figure, the purpose of which is to illustrate the invention rather than to limit its scope.

### BRIEF DESCRIPTION OF THE DRAWING

In the appended drawing:

FIG. 1 shows the immunohistochemical staining of prostate cancer tissues ×400. Panels A to E show the localization and staining of NF-κB transcription factors. Panel A shows RelA/p65 (Santa Cruz Biotech., SC-8008) cytoplasmic and nuclear staining. Panel B shows RelA/p65 (NeoMarkers, RB-1638) cytoplasmic and nuclear staining. Panel C shows NF-κB1/p50 cytoplasmic and nuclear staining. Panel D shows RelB cytoplasmic and nuclear staining. Panel E shows NF-κB2/p52 cytoplasmic and some nuclear staining. Panel F shows c-Rel cytoplasmic staining. With the exception of c-Rel, all can be localized in the nuclei of cancer cells.

The following embodiments are described referring to a body of literature, the contents of which are incorporated by reference.

### DESCRIPTION OF ILLUSTRATIVE EMBODIMENTS

The present invention is illustrated in further details by the following non-limiting examples.

Immunohistochemical Detection of NF-κB

Specimens were fixed with formalin and embedded in paraffin (frozen tissue sections can also be used), sectioned, and stained by an immunoperoxidase method. Any suitable staining method can be used. For example, cell fractions comprising a nuclear fraction on one hand and a cytoplasmic fraction on the other one hand could be separately tested for the presence of NF-kB. The presence of NF-kB can be revealed by binding to a NF-kB ligand, and complexes NF-kB/ligand can be revealed by staining the same (without being so limited the alkaline phosphatase method for instance could also be used as a staining method) or by labeling the ligand with a fluorescent or radioactive molecule. “Staining” is not limited to the use of dyes; it also includes any reactant permitting the observation and distinction between cells and nuclei, and the quantification of NF-kB present in both compartments. Tissue sections were immunostained for the NF-κB p65 transcription factor using 3 antibodies recognizing different regions of the p65 protein (see table 1 below for a complete list of all p65 antibodies available): 1) the NF-kB p65(F-6) mouse monoclonal antibody (Santa Cruz Biotechnology, Calif.) which recognizes amino-terminal sequences of the p65 subunit, 2) the NF-κB p65 (RelA) Ab-1 rabbit polyclonal antibody (Neo Markers, Lab Vision, Calif.) recognizing an epitope localized in the C-terminal region of p65, and 3) the NF-κB p65 epitope specific antibody (Neo Markers, Lab Vision, Calif.) that recognizes an internal domain of p65. The other NF-κB subunits were detected with the following antibodies: rabbit polyclonal RelB c-19, mouse monoclonal c-Rel B6, rabbit polyclonal NF-κB p50 NLS (all from Santa Cruz Biotech, Santa Cruz, Calif.), and NF-κB p52 (Upstate Biotechnology Inc, Lake Placid, N.Y.).

TABLE 1
Commercially Available NF-kB p65 Antibodies

| Company | Antibody | Description | Specificity |
|---|---|---|---|
| Abcam | NF-kB (p65) antibody (ab7970) | Rabbit polyclonal | Carboxy terminal domain of human NF-kB p65 |
| Abcam | NF-kB (p65) antibody (ab243) | Sheep polyclonal | Full length human p65 |
| Abcam | NF-kB (p65) antibody (ab7552) | Rabbit polyclonal | N-terminal region |
| Abcam | NF-kB antibody (ab2615) | Rabbit polyclonal | Recognises the phospho peptide |
| Abcam | NF-KB antibody (ab2106) | Rabbit polyclonal | Near the C-terminus of the human protein |
| Active Motif | NF-kB p65 (40916) | Mouse Monoclonal | Amino acid residues 529-536 |
| Active Motif | NF-kB p65 (39038) | Rabbit polyclonal | Amino acid residues 2-17 |
| Active Motif | NF-kB p65 (39039) | Rabbit polyclonal | Amino acid residues 501-515 |
| Active Motif | NF-kB p65 (39331) | Rabbit polyclonal | Amino acid residues 2-17 |
| Assay Designs Inc. | NF kappa B p65 C (91522) | Rabbit | C-terminal region |
| Assay Designs Inc. | NF kappa B p65 C (91525) | Rabbit | N-terminal region |
| BD Bioscience | NF-kB p65 (610868) | Mouse | Amino acid residues 136-224 |
| Cell Signalling | NF kappa B p65 (3034) | Rabbit polyclonal | Amino acid residues around S276 |
| Cell Signalling | NF kappa B p65 S536 (3031) | Rabbit polyclonal | Phospho-S536 |
| Chemicon | NF kappa B p65 (MAB3026) | Mouse Monoclonal | p65 NLS |
| Chemicon | NF kappa B p65 (AB1604) | Rabbit polyclonal | C-terminal region |
| Fitzgerald | p65 RelA 20-PR05 | Rabbit polyclonal | N/A |
| Imgenex | p65 (IMG512) | Rabbit polyclonal | N/A |
| Neo Markers | NF kappa B p65 Ab-1 (RB-1638) | Rabbit polyclonal | C-terminal region |
| Neo Markers | NF-kappa B (RB-9034) | Rabbit | Internal domain |
| Oncogene Research Products | p65 (RelA) Ab-2 (PC-138) | Rabbit polyclonal | N-terminal |
| Oncogene Research Products | p65 (RelA) Ab-1 (PC-137) | Rabbit polyclonal | C-terminal region |
| Rockland Immunochemicals | p65 (100-4165N) | Rabbit | N-terminal |
| Rockland Immunochemicals | NF kappa B p65 S276 (100-401-264) | Rabbit | Phospho-S276 |
| Rockland Immunochemicals | NF kappa B p65 S529 (100-401-266) | Rabbit | Phospho-S529 |
| Santa Cruz Biotechnology | NF-kB p65 A (sc-109) | Rabbit polyclonal | N-terminal region |
| Santa Cruz Biotechnology | NF-kB p65 C-20 (sc-372) | Rabbit polyclonal | C-terminal region |
| Santa Cruz Biotechnology | NF-kB p65 H-286 (sc-7151) | Rabbit polyclonal | N-terminal region |
| Santa Cruz Biotechnology | NF-kB p65 F-6 (sc-8008) | Mouse Monoclonal | N-terminal region |
| Sigma | NF-kB p65 (N 8523) | Mouse Monoclonal | C-terminal region |
| Spring Bioscience | NF kappa B p65 (E2751) | Rabbit | Internal domain |
| Upstate Biotechnology | NF-kB p65 CT (06-418) | Rabbit polyclonal | C-terminal region |
| Zymed | NF-kB p65 (33-9900) | Mouse Monoclonal | C-terminal region |

Initially, tissue sections were deparaffinized with toluene and rehydrated through graded ethanol. All steps were performed at room temperature. Following each step, sections were washed with 0.01M phosphate buffered saline (PBS solution) for 10 min. Endogenous peroxidase activity was blocked with 3% hydrogen peroxide for 10 min. Tissue sections were incubated with a protein blocking serum-free reagent (Dako Diagnostics, Inc.,Ont.,Canada) for 15 min to block non-specific binding. An antigen retrieval technique was applied by boiling slides for 10 min at 95° C. in a 0.01M sodium citrate buffer pH 6.0 (for RelA, RelB, p50, and p52 staining) or 1 mM EDTA pH 8.0 (for c-Rel staining). Primary antibodies were applied at a concentration of 1:10 (c-Rel) or 1:50 (RelA, RelB, p50, p52) in PBS and were incubated for 120 min at room temperature. Immune complexes were revealed using a biotin-conjugated broad spectrum secondary antibody (20 min), then streptavidin-peroxidase conjugate for 20 min (DakoCytomation, Denmark), followed by chromogen (0.06% 3,3-diaminobenzidine tetrahydrochloride, 0.01% hydrogen peroxide in PBS) for 5 min. Sections were counter stained with Mayer's haematoxylin, dehydrated, and then mounted. Negative controls were included by omitting the primary antibody. Positive staining was observed by light microscopy and localized by brown staining within the cell cytoplasm and/or nucleus. The overall percentage of NF-κB-nuclear localization was assessed, as well as the intensity of staining. All slides were independently analyzed in a blinded study. Alternatively, the immunohistochemical analysis of NF-κB transcription factors can be performed on frozen tissue sections. Tissues are fixed in cold acetone-ethanol (3:1) 10 min and rinsed in PBS. The following steps are identical to those of formalin-fixed paraffin-embedded tissues.

### EXAMPLE 1

NF-κB can Predict the Development of Prostate Cancer Bone Metastases

Detection of NF-κB(p65) in Prostate Cancer

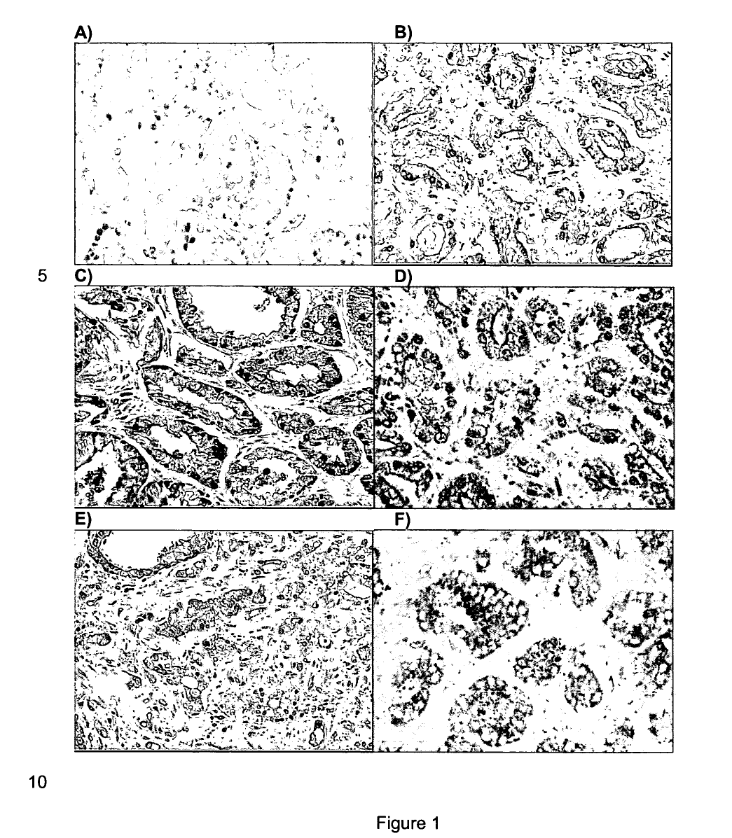

In our first study, all 45 tumors demonstrated cytoplasmic staining and 18/45 tumors also had positive nuclear staining. NF-κB was detected in the cytoplasm of normal and tumor cells, but nuclear localization was observed only in cancer cells (example of p65 nuclear staining in FIG. 1a). In normal glands surrounding the tumors, basal cells had higher intensity of staining than luminal cells.

We tested whether nuclear NF-κB was linked to the degree of histological differentiation. Tumors were analyzed according to Gleason grade and were sub-divided into three groups. Nine tumors were well differentiated (Gleason scores 2-4), 24 showed moderate differentiation (Gleason scores 5-7), and 12 tumors were classified as poorly differentiated (Gleason scores 8-10). Although NF-κB nuclear staining was observed mainly in moderately and poorly differentiated tissues, NF-κB nuclear localization and the Gleason score were not linked (p=0.089). Overall, NF-κB nuclear localization is observed in prostate cancer and appears to be an independent marker from the Gleason score.

NF-κB (p65) Nuclear Localization and Prognosis

In the second study, tissue specimens from 30 patients with known outcomes of prostate adenocarcinoma were selected. Seventeen and thirteen specimens were included in the poor and good outcome groups respectively. A poor outcome was defined as recurrence following radical prostatectomy and progression to bone metastases (positive bone scan), whereas a good outcome was defined as no evidence of cancer recurrence (PSA=0) for a minimum of 5 years post-prostatectomy. All specimens showed some degree of immunostaining. Tissues were sub-divided into three groups: 5 tumors were well differentiated (Gleason scores 2-4), 17 showed moderate differentiation (Gleason scores 5-7), and 8 tumors were classified as poorly differentiated (Gleason scores 8-10). As expected, there was a significant association (p=0.05) between prognosis and increasing histological grade. However, it is hard to predict patient outcome in the presence of moderately differentiated tissues (Gleason scores 5-7). Over 50% of cancers in both the good and poor outcome groups had a Gleason score in the 5-7 category. The use of an additional prognostic marker would be useful to predict patient outcome (Data not shown).

We tested whether NF-κB nuclear localization correlated with prognosis. Using negative and positive NF-κB nuclear staining as the sole prediction parameter does not help predetermine patient outcome (p=0.413). However, when we classify nuclear NF-κB immunostaining into groups having 0-10% and >10% tumor staining, NF-κB nuclear localization relates to patient outcome. Fifty-nine percent (10/17) of patients with a poor outcome had positive nuclear staining in over 10% of the tumor surface, whereas only 15% (2/13) of the sample from the good outcome group were positive in over 10% of the tumor surface (p=0.026).

As with our initial analysis, the Gleason score and the nuclear localization of NF-κB, are independent (p=0.262). The Gleason score and NF-κB nuclear localization are independent markers, yet both correlate with patient outcome. We therefore reclassified tumor samples into two risk categories: low risk and high or significant risk for cancer progression. The low risk category includes all Gleason scores of 2-4, and Gleason scores 5-7 with less than 10% nuclear staining. The high risk category includes all Gleason scores 8-10, as well as scores 5-7 with over 10% nuclear staining. Using this stratification method, 85% (11/13) of specimens in the good outcome group are correctly assigned to the low risk category (p=0.004). Similarly, 71% (12/17) of specimens in the poor prognosis group are accurately predicted to have a high risk of progression. Although the cut-off value above which the NF-kB staining is considered positive has been hereinabove fixed at 10%, this minimal value is to be varied between 5 to 20% to optimize the reliability and the accuracy of the test, also taking into consideration what type of cancer progression or recurrence is under evaluation.

Overall, NF-κB expression is detectable mainly in basal cells of normal prostate glands and in prostate cancer tissues. Nuclear localization of NF-κB is detectable in prostate cancer tissues but it does not correlate with the degree of histological differentiation. We have demonstrated that NF-κB can be useful, in combination with the Gleason score, to stratify patients into low and high risk categories of cancer progression. These results justify continuing a larger scale study analyzing NF-κB as a prognostic marker. NF-κB may eventually aid in tailoring therapy according to risk categories.

### EXAMPLE 2

NF-κB can Predict the Presence/Development of Prostate Cancer Lymph Node Metastases

Seventy-seven paraffin-embedded lymph node specimens obtained from 54 prostate cancer patients were analyzed. Of the 54 patients, 32 had positive lymph node metastases, while 22 showed no evidence of metastasis and were considered as controls. Nuclear localization of NF-κB was significantly greater in the metastatic lymph node group compared to controls. In patients with positive-lymph node metastases, 84.4% showed >10% nuclear staining in tumor cells. Moreover, 64.4% of the malignant lymph node specimens had >10% nuclear staining in lymphocytes compared to 0% in controls. Intensity of cytoplasmic and nuclear staining was higher in the metastatic lymph node group than in controls (p<0.01).

When comparing the 32 lymph node metastases with their corresponding primary prostate cancer tumors, we observed a concomitant up-regulation of nuclear NF-κB. This suggests that the constitutive activation of NF-κB in clinically localized prostate cancer can predict the presence/development of lymph node metastases.

### EXAMPLE 3

NF-κB can Predict Biochemical Relapse After Radical Prostatectomy

Radical prostatectomy is the most common treatment option for men with clinically localized disease. For those patients who manifest a pathological evidence of post-operative positive surgical margins (pT3), it is still controversial whether aggressive adjuvant treatment should be instituted or not. We examined the sub-cellular localization of NF-κB in tissues of pathologically proven prostate cancer with positive surgical margins of prospectively followed patients and correlated it with clinical outcome.

Four (4) years after surgery, patients with negative (<5%) nuclear NF-κB tumors had an 80% chance of being free of biochemical recurrence (detectable PSA after radical prostatectomy) as opposed to those with positive (>5%) nuclear NF-κB tumors which has only a 38% chance of relapse (p=0.034). Overall, NF-κB sub-cellular localization is correlated with a biochemical relapse. This may help tailoring adjuvant therapy according to risk categories, in this group of patients for which any therapeutic decision is, up to now, controversial.

### EXAMPLE 4

NF-κB can Predict the Development of Hormone-Refractory Prostate Cancer

All the patients from which tissue samples were obtained for this study had hormone-refractory prostate cancer, as defined by local and/or systemic progression under adequate antiandrogen or LHRH agonist therapy, or a combination of both. Transurethral Radical Prostatectomy (TURP) was performed for patient with urinary obstruction in order to improve quality of life. Twenty-four such male patients, aged 55-83 years, required TURP between 1992 and 2001. Adequate specimen was available for 20 patients. The mean Gleason score was 8.5 (range 7-10).

In 8 samples, the NF-κB staining was almost exclusively (>90%) nuclear. Two tissue samples demonstrated NF-κB staining homogenously distributed between the nucleus and the cytoplasm. The remaining samples (10) presented an almost exclusively cytoplasmic pattern. The presence of nuclear NF-κB in 60% of hormone-refractory specimens analyzed is indicative of a favorable role of NF-κB activation in the progression of prostate cancer to an androgen-independent disease.

Sub-Cellular Localization of Other NF-κB Subunits in Prostate Cancer

Immunohistochemical analyses also demonstrated that all NF-κB transcription factors are expressed in prostate tissues. With the exception of c-Rel, all can be localized in the nuclei of cancer cells (see FIG. 1, panels A to E). Therefore, except for c-Rel, NF-κB subunits and not only p65 can potentially be used alone and/or together as prognostic markers in prostate cancer.

The invention being hereinabove described, it will be obvious that the same may be varied in many ways. Those skilled in the art recognize that other and further changes and modifications may be made thereto without departing from the spirit of the invention, and it is intended that all such changes and modifications fall within the scope of the invention, as defined in the appended claims.

References

1. Abate-Shen, C. and M. M. Shen, Molecular genetics of prostate cancer. Genes Dev, 2000.14(19): p. 2410-34.
2. Nelson, W. G., A. M. De Marzo, and W. B. Isaacs, Prostate cancer. N Engi J Med, 2003. 349(4): p. 366-81.
3. Roylance, R., N. Spurr, and D. Sheer, The genetic analysis of prostate carcinoma. Sem. Cancer Biol., 1997. 8(1): p. 37-44.
4. Lalani el, N., M. E. Laniado, and P. D. Abel, Molecular and cellular biology of prostate cancer. Cancer Metast. Rev., 1997.16(1-2): p. 29-66.
5. Bruckheimer, E. M. and N. Kyprianou, Apoptosis in prostate carcinogenesis. A growth regulator and a therapeutic target. Cell Tissue Res, 2000. 301(1): p. 153-62.
6. Johnson, M. I. and F. C. Hamdy, Apoptosis regulating genes in prostate cancer (review). Oncol. Reports, 1998. 5(3): p. 553-7.
7. Baldwin, A. S., Control of oncogenesis and cancer therapy resistance by the transcription factor NF-kappaB. J Clin Invest, 2001. 107(3): p. 241-6.
8. Karin, M., et al., NF-kappaB in cancer: from innocent bystander to major culprit. Nature Rev Cancer, 2002. 2(4): p. 301-10.
9. Sovak, M. A., et al., Aberrant nuclear factor-kappaB/Rel expression and the pathogenesis of breast cancer. J Clin Invest, 1997. 100(12): p. 2952-60.
10. Wang, W., et al., The nuclear factor-kappa B RelA transcription factor is constitutively activated in human pancreatic adenocarcinoma cells. Clin Cancer Res, 1999. 5(1): p.119-27.
11. Sumitomo, M., et al., An essential role for nuclear factor kappa B in preventing TNF-alpha-induced cell death in prostate cancer cells. J Urol, 1999. 161(2): p. 674-9.
12. Flynn, V., Jr., et al., Adenovirus-mediated inhibition of NF-kappaB confers chemo-sensitization and apoptosis in prostate cancer cells. Int J Oncol, 2003. 23(2): p. 317-23.
13. Suh, J. and A. B. Rabson, NF-kappaB activation in human prostate cancer:
   important mediator or epiphenomenon?J Cell Biochem, 2004. 91 (1): p. 100-17.
14. Palayoor, S. T., et al., Constitutive activation of IkappaB kinase alpha and NF-kappaB in prostate cancer cells is inhibited by ibuprofen. Oncogene, 1999. 18(51): p. 7389-94.
15. Chen, C. D. and C. L. Sawyers, NF-kappa B activates prostate-specific antigen expression and is upregulated in androgen-independent prostate cancer. Mol Cell Biol, 2002. 22(8): p. 2862-70.
16. Gasparian, A. V., et al., The role of IKK in constitutive activation of NF-kappaB transcription factor in prostate carcinoma cells. J Cell Sci, 2002. 115(Pt 1): p. 141-51.
17. Suh, J., et al., Mechanisms of constitutive NF-kappaB activation in human prostate cancer cells. Prostate, 2002. 52(3): p. 183-200.
18. Huang, S., et al., Blockade of NF-kappaB activity in human prostate cancer cells is associated with suppression of angiogenesis, invasion, and metastasis. Oncogene, 2001. 20(31): p. 4188-97.
19. Ling, M. T., et al., Id-1 expression promotes cell survival through activation of NF-kappaB signalling pathway in prostate cancer cells. Oncogene, 2003. 22(29): p. 4498-508.
20. Hodge, J. C., et al., Requirement of RhoA activity for increased nuclear factor kappaB activity and PC-3 human prostate cancer cell invasion. Cancer Res, 2003. 63(6): p.1359-64.
21. Levine, L., et al., Bombesin stimulates nuclear factor kappa B activation and expression of proangiogenic factors in prostate cancer cells. Cancer Res, 2003. 63(13): p. 3495-502.
22. Rayet, B. and C. Gelinas, Aberrant rel/nfkb genes and activity in human cancer. Oncogene, 1999.18(49): p. 6938-47.
23. Lessard, L., et al., NF-kappa B nuclear localization and its prognostic significance in prostate cancer. BJU Int, 2003. 91(4): p. 417-20.
24. Ismail, H. A., et al., Expression of NF-kappaB in prostate cancer lymph node metastases. Prostate, 2004. 58(3): p. 308-13.
25. Gerondakis, S., et al., Genetic approaches in mice to understand Rel/NF-kappaB and IkappaB function: transgenics and knockouts. Oncogene, 1999. 18(49): p. 6888-95.
26. Hu, Y., et al., Abnormal morphogenesis but intact IKK activation in mice lacking the IKKalpha subunit of IkappaB kinase. Science, 1999. 284(5412): p. 316-20.
27. Tanaka, M., et al., Embryonic lethality, liver degeneration, and impaired NF-kappa B activation in IKK-beta-deficient mice. Immunity, 1999. 10(4): p. 421-9.
28. Egan, L. J., et al., I{kappa}B-kinase{beta}-dependent NF-{kappa}B activation provides radioprotection to the intestinal epithelium. Proc Natl Acad Sci U S A, 2004.

1. A method for predicting whether a patient suffering from a prostate cancer is at risk of experiencing a cancer progression or recurrence which comprises the steps of:
obtaining a Gleason score of said prostate tumor sample;
assessing the proportion of NF-kB localized in the nuclei of said tumor sample with regard to all NF-kB present in said tumor sample.

2. A method for preparing a prostate cancer sample for predicting whether a patient suffering from a prostate cancer is at risk of experiencing a cancer progression or recurrence which comprises the steps of:
obtaining a Gleason score of said prostate tumor sample;
staining the cells and the nuclei of a prostate tumor sample obtained from said patient;
staining the cells and the nuclei for the presence of NF-kB.

3. A method as recited in claim 2 further comprising calculating a proportion of staining of NF-kB localized in the nuclei with regard to the total cell staining of NF-kB in a given surface area of tumor sample.

4. A method for predicting whether a patient suffering from a prostate cancer is at risk of experiencing a cancer progression or recurrence which comprises the steps of:
staining the cells and the nuclei of a prostate tumor sample obtained from said patient;
obtaining a Gleason score of said prostate tumor sample;
staining the cells and the nuclei for the presence of NF-kB;
calculating a proportion of staining of NF-kB localized in the nuclei with regard to the total cell staining of NF-kB in a given surface area of tumor sample,
wherein about 5% or more of the NF-kB staining in said surface being localized in the cell nuclei and a Gleason score higher than 5 are indicative that the patient is at risk of experiencing cancer progression or recurrence.

5. The method of claim 4, wherein about 10% or more of the NF-kB staining in said surface localized in the cell nuclei is indicative that the patient is at risk of experiencing cancer progression or recurrence.

6. The method of claim 4, wherein about 20% or more of the NF-kB staining in said surface localized in the cell nuclei is indicative that the patient is at risk of experiencing cancer progression or recurrence.

7. The method of claim 4, wherein said cancer progression or recurrence is identified through biochemical recurrence, lymph node metastases, bone metastases, and/or hormone refractoriness.

8. The method of claim 4 wherein the presence of NF-kB is revealed with a ligand to NF-kB p65 epitope.

9. The method of claim 8, wherein the ligand is a monoclonal antibody specific to the C-terminal region of p65.

10. A diagnostic kit for predicting a risk incurred by a patient suffering from a prostate cancer of experiencing a cancer progression or recurrence, which comprises:
reactants for staining the cells and nuclei of a prostate tumor sample, and
reactants for staining the cells and nuclei for the presence of NF-kB.

11. The diagnostic kit of claim 10, wherein the reactants for staining the cells and nuclei for the presence of NF-kB comprise a ligand to NF-KB p65 epitope.

12. The diagnostic kit of claim 11, wherein the ligand is a monoclonal antibody to the C-terminal region of p65.
